(19) United States
(12) Patent Application Publication
(10) Pub. No.: US 2007/0258874 A1
(43) Pub. Date: Nov. 8, 2007

(54) Sanitizer for manicure tools

(76) Inventor: Hai Vuong, Swedesboro, NJ (US)

Correspondence Address: Hollstein Keating Cattell Johnson & Goldstein P.C.;Willow Ridge Executive Office Park, Suite 301, 750 Route 73 South, Marlton, NJ, 08053, US

(21) Appl. No.: 11/416,697

(22) Filed: May 3, 2006

(51) Int. Cl. A61L 2/18

(52) U.S. Cl. 422/300

(57) ABSTRACT

A sanitizer for manicure tools has a container into which sanitizing fluid is stored. A manicure tool holder receives and secures various types of manicure tools. The holder is configured to move vertically, raising and lowering the tools into and out of the fluid. A push button activates a click-type ballpoint pen operating system to position the holder, carrying its manicure tools, from a first, distinct stationary position in which the tools are out of the fluid, to a second distinct, stationary position, with the tools being located in the fluid for cleaning and sanitizing purposes. The cover of the container, the manicure tool holder and the operating system make up a removable manicure tool holder unit which permits the tools to be lifted out of the container.

### BACKGROUND OF THE INVENTION

There are a wide variety of manicuring tools which are used in the commercial, nail manicure trade, each with a specific function and purpose. In the normal course of business, the manicurist will use the same tools to serve many different customers. As a result, the tools must be cleaned periodically, in order to ensure for the necessary sanitary conditions which are required. However, it is often not practical to sanitize manicuring tools adequately between uses, especially during busy periods. Tools sometimes are used a number of times before they are cleaned or, when they are cleaned, they are not cleaned sufficiently. Acquiring many duplicate sets of tools so that sets can be used while others are being cleaned is expensive. The solution to the problem is the development of a quick and easy means of sanitizing manicure tools, which allows the tools to be efficiently positioned for cleaning, which aligns them for easy cleaning, and which permits easy access to clean tools when they are needed.

### SUMMARY OF THE INVENTION

It is the object of the present invention to address the limitations and disadvantages presented by the use of unsanitized manicure tools.

It is thus an object of the present invention to provide a sanitizer for manicure tools which provides a safe and effective means of quickly cleaning and sanitizing manicure tools.

It is a further object of the present invention to provide a sanitizer for manicure tools which allows manicure tools to be easily positioned in a sanitizer for cleaning, and permits ready retrieval of the tools for use.

It is another object of the present invention to provide a sanitizer for manicure tools which employs a container for storing sanitizing fluid, a manicure tool holder positioned within the container, and a click-type ball point pen operating system to quickly and efficiently raise and lower manicure tools in the sanitizing fluid within the holder.

These and other objects are accomplished by the present invention, a sanitizer for manicure tools which comprises a container into which sanitizing fluid is stored. A manicure tool holder receives and secures various types of manicure tools. The holder is configured to move vertically, raising and lowering the tools into and out of the fluid. A push button activates a click-type ballpoint pen operating system to position the holder, carrying its manicure tools, from a first, distinct stationary position in which the tools are out of the fluid, to a second distinct, stationary position, with the tools being located in the fluid for cleaning and sanitizing purposes. The cover of the container, the manicure tool holder and the operating system make up a removable manicure tool holder unit which permits the tools to be lifted out of the container.

Novel features which are considered as characteristic of the invention are set forth in particular in the appended claims. The invention itself, both as to its design, construction and use, together with the additional features and advantages thereof, are best understood upon review of the following detailed description with reference to the accompanying drawings.

### BRIEF DESCRIPTION OF THE DRAWINGS

FIG. 1 is an elevation view of the manicure tool sanitizer of the present invention with its manicure tool holder in the raised position.

FIG. 2 is an elevation view of the manicure tool sanitizer of the present invention with its manicure tool holder in the lowered position.

FIG. 3 is a cross-sectional view of the operating system showing the manicure tool holder in the raised position.

FIG. 4 is a cross-sectional view of the operating system showing the manicure tool holder in the lowered position.

### DETAILED DESCRIPTION OF THE INVENTION

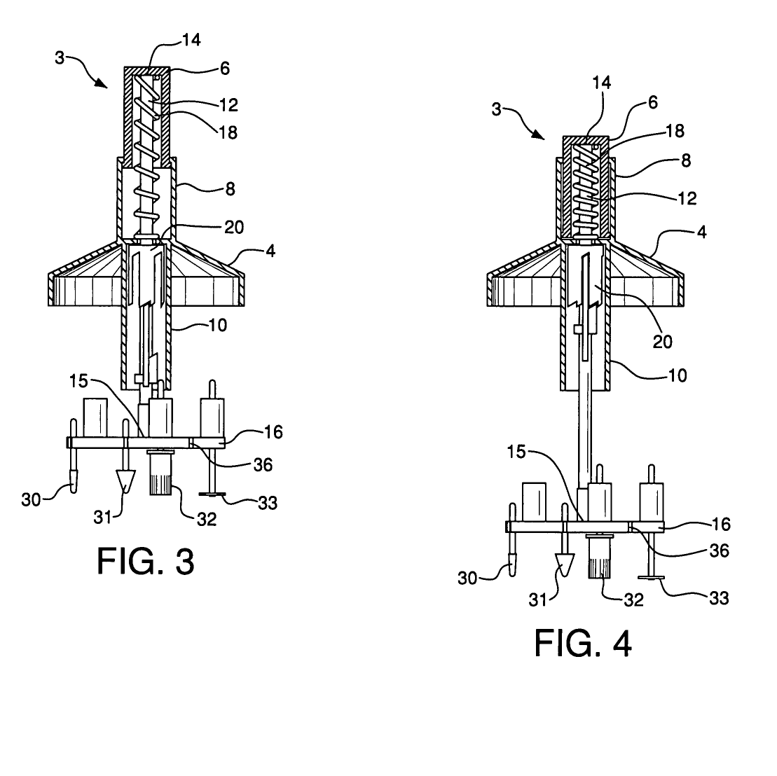

Manicure tool sanitizer 1 of the present invention comprises container 2, which is preferably clear glass or plastic, but could be of any similar, suitable material. Manicure tool holder unit 3, which includes an actuating mechanism, shown in FIGS. 3 and 4, is completely removeable from container 2. Unit 3 comprises cover 4 configured to be positioned on top of container 2. Push button 6, located partially within upper housing 8 extending from cover 4, is configured to actuate a conventional, click-type ballpoint pen mechanically operable system. In the embodiment which is shown, this system is enclosed by upper housing 8 and lower housing 10. Shaft 12 is connected, at its upper end 14, to the internal top surface of button 6 and, at its lower end 15, to manicure tool holder 16. Spring 18, surrounding shaft 12, biases the shaft upward against button 6, maintaining holder 16 in a raised position, within container 2, as shown in FIGS. 1 and 3. Rotatable plunger actuation mechanism 20, common to click-type ballpoint pen operations, is positioned around shaft 12. When button 6 is actuated, i.e. pushed, shaft 12 is pushed down against the bias of spring 18. Holder 16, connected to shaft 12 at lower end 15, is then moved vertically downward into container 2. Plunger mechanism 20, rotatably operating as it would to click a ball-point pen in place, similarly clicks holder 16 into this lowered position within container 2. Pushing button 6 a second time, clicks plunger mechanism 20, again rotating it to release shaft 12, causing spring 18 to biasedly expand, moving the shaft and holder 16 vertically upwards within container 2.

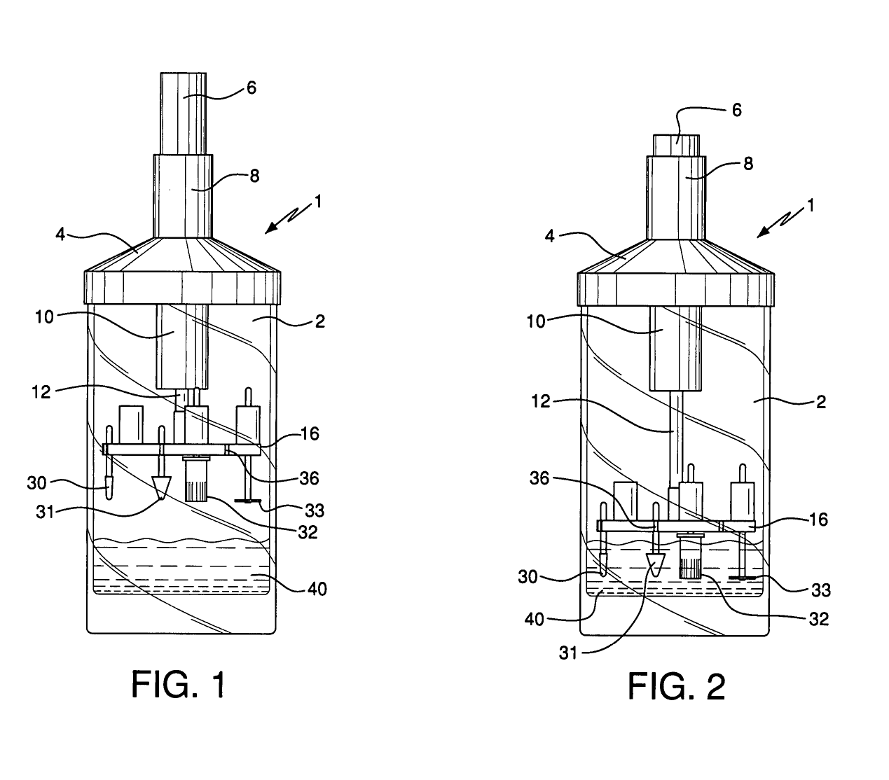

Various soiled manicure tools 30, 31, 32, and 33 are secured within holder 16, while unit 3 is removed from container 2. Unit 3 is then placed on and within container 2 by positioning cover 4 over the container. This placement, as shown in FIG. 1, positions holder 16 and the various manicure tools 30-33 secured within the holder, in a stationary, raised position above sanitizing fluid 40. By pushing button 16, holder 16 and the various manicure tools 30-33 within the holder, are lowered into sanitizing fluid 40, as shown in FIG. 2. Plunger mechanism 20 clicks holder 16 and locks it into a distinct, stationary position within fluid 40, allowing tools 30-33 to be cleaned and sanitized. Holder 16 and tools 30-33 remain in fluid 40 until the holder and its tools are raised out of the fluid, by pressing button 6 to again click the holder back into the distinct, stationary raised position shown in FIG. 1. Unit 3 can then be removed from container 2 so that the sanitize tools can be removed and replaced with tools to be cleaned.

Holder 16, as shown, comprises a plurality of notches 36, which serve to lock manicure tools 30-33 into place. Other equivalent tool to holder attachment means can be used and the invention is not to be considered restricted to the manner of this attachment.

Similarly, while one example of a click-type ballpoint pen operating system is described to click and position holder 16 into its stationary raised and lowered positions, the invention is not to be considered restricted to this system. Any known, readily adaptable click-type ballpoint pen operating system can be used for this purpose.

These and other novel features and components of this invention are disclosed in detail in order to make the invention clear in at least one form thereof. However, it is to be clearly understood that the invention as disclosed is not necessarily limited to the exact form and details as disclosed, since it is apparent that various modifications and changes may be made without departing from the spirit of the invention.

1. A sanitizer for manicure tools comprising:
base means for supporting manicure tools;
connection means to maintain and secure the manicure tools on the base means;
container means for retaining a sanitizing fluid, said container means enclosing the base means and the manicure tools on the base means;
actuating means to initiate movement of the base means vertically within the container means;
mechanically operable drive means connected between the actuating means and the base means for moving the base means vertically solely between two distinct stationary positions within the container means and for clicking the base means from one stationary position to the other stationary position, whereby upon operation of the actuating means, the drive means moves the base means vertically into and out of the sanitizing fluid and alternately clicks the base means into the two stationary positions, locking the base means into these positions.

2. The sanitizer as in claim 1 wherein the connection means comprises a plurality of openings within the base means through which the manicure tools are maintained and secured.

3. The sanitizer as in claim 1 wherein the two stationary positions comprise an up position out of the sanitizing fluid and a down position within the sanitizing fluid.

4. The sanitizer as in claim 3 wherein the drive means comprises means to click the base means into the up and down positions.

5. The sanitizer as in claim 1 wherein the drive means comprises push button, ballpoint pen operating means for moving the base means solely between the two distinct stationary positions within the container means and to alternately click the base means into the two stationary positions.

6. The sanitizer as in claim 5 wherein the positions comprise an up position out of the sanitizing fluid and a down position within the sanitizing fluid.

7. The sanitizer as in claim 5 wherein the ballpoint pen operating means comprises means to click the base means into the up and down positions.

8. The sanitizer as in claim 1 wherein the drive means comprises means to alternately click the base means solely into the two stationary positions within the container means.

9. The sanitizer as in claim 8 wherein the positions comprise an up position out of the sanitizing fluid and a down position within the sanitizing fluid.

10. The sanitizer as in claim 1 where the actuating means comprises push button means for the application of manual pressure, whereby upon application of manual pressure on the push button means, the drive means moves the base means vertically and clicks the base means into one of the two distinct stationary positions within the container means.

11. The sanitizer as in claim 1 wherein the drive means comprises a ballpoint pen click type operating mechanism.

12. The sanitizer as in claim 5 wherein the ballpoint pen operating means comprises a resiliently biased support arm moved vertically up and down within the container by click actuation linkage.

13. The sanitizer as in claim 10 wherein the drive means comprises a resiliently biased support arm moved vertically up and down within the container by click actuation linkage, whereby upon application of manual pressure on the push button means, the click actuation linkage compels the support arm to move, thereby clicking the base means from stationary position to stationary position.

14. The sanitizer as in claim 1 wherein the base means, the connection means, the actuating means, and the drive means are configured as a single component which is removeable and separable from the container means.

15. A sanitizer for manicure tools comprising:
container means for retaining a sanitizing fluid, and
separate manicure tool holder means configured to be positioned on and to be removed from the container means, said holder means comprising base means for supporting manicure tools, connection means to maintain and secure the manicure tools on the base means, actuating means to initiate vertical movement of the base means, and mechanically operable drive means connected between the actuating means and the base means for moving the base means vertically solely between two distinct stationary positions and for clicking the base means from one stationary position to the other stationary position, whereby when the holder means is positioned on the container means and upon operation of the actuating means, the drive means moves the base means vertically into and out of the sanitizing fluid and alternately clicks the base means into the two stationary positions, locking the base means into these positions.

16. The sanitizer as in claim 15 wherein the drive means comprises push button, ballpoint pen operating means for moving the base means solely between the two distinct stationary positions within the container means and to alternately click the base means into the two stationary positions.

17. The sanitizer as in claim 15 wherein the actuating means comprises push button means for the application of manual pressure, whereby upon application of manual pressure on the push button means, the drive means moves the base means vertically and clicks the base means into one of the two distinct stationary positions within the container means.

18. The sanitizer as in claim 15 wherein the drive means comprises a ballpoint pen click type operating mechanism.

19. The sanitizer as in claim 16 wherein the ballpoint pen operating means comprises a resiliently biased support arm moved vertically up and down within the container by click actuation linkage.

20. The sanitizer as in claim 15 wherein the drive means comprises a resiliently biased support arm moved vertically up and down within the container by click actuation linkage, whereby application of manual pressure on the push button means, the click actuation linkage compels the support arm to move, thereby clicking the base means from stationary position to stationary position.
